(19) United States
(12) Patent Application Publication
(10) Pub. No.: US 2009/0069768 A1
(43) Pub. Date: Mar. 12, 2009

(54) Disposable Undergarment

(76) Inventor: Brenda A. Hunt, Jacksonville, FL (US)

Correspondence Address: HARSHAW RESEARCH, INC., 210 W. TECUMSEH STREET, OTTAWA, KS, 66067, US

(21) Appl. No.: 11/854,165

(22) Filed: Sep. 12, 2007

(51) Int. Cl. A61F 13/15

(52) U.S. Cl. 604/385.01; 604/396; 604/398; 604/399

(57) ABSTRACT

A disposable undergarment according to the present invention includes an outer element having a waistband and defining two leg holes, the outer element presenting inner and outer surfaces. The disposable undergarment includes an absorbent inner element removably coupled to the outer element inner surface. The inner element is disposable in a toilet and the outer element is disposable as solid refuse. The inner element is removably coupled to the outer element with an adhesive. In addition, the inner element may include a plurality of absorbent layers. The outer element waistband may include fasteners for separating the outer element into front and rear portions.

### BACKGROUND OF THE INVENTION

This invention relates generally to undergarments and, more particularly, to a disposable undergarment that assists with absorbing a women's menstrual flow.

It is typical for a woman to wear a moisture absorbent pad in her underwear during the flow of her monthly menstrual cycle. Although a maxi-pad is effective to prevent the wetness of the menstrual flow from absorbing into a woman's underpants or leaking therefrom onto the woman's clothing, it may be very inconvenient for a woman to carry additional pads with her throughout the day or to go through the process of disposing of the pads and positioning a new one in her underpants. In specific occupations or activities, it may be difficult to maintain a pad in a desired position in the underpants.

Various devices have been proposed for absorbing the menstrual flow of a woman's monthly cycle. Although assumably effective for their intended purposes, the existing proposals do not provide the functionality of a maxi-pad while still having the attractiveness of high quality underwear/panties.

Therefore, it would be desirable to have a disposable undergarment having the effective absorbency of a maxi-pad as well as the aesthetic attractiveness of a traditional undergarment. Further, it would be desirable to have a disposable undergarment having an absorbent layer that is removable from the undergarment and disposable in a toilet. In addition, it would be desirable to have a disposable undergarment in which the remainder of the undergarment is disposable in a traditional manner.

### SUMMARY OF THE INVENTION

Accordingly, a disposable undergarment according to the present invention includes an outer element having a waistband and defining two leg holes, the outer element presenting inner and outer surfaces. The disposable undergarment includes an absorbent inner element removably coupled to the outer element inner surface. The inner element is disposable in a toilet and the outer element is disposable as solid refuse. The inner element is removably coupled to the outer element with an adhesive. In addition, the inner element may include a plurality of absorbent layers. The outer element waistband may include fasteners for separating the outer element into front and rear portions.

Therefore, a general object of this invention is to provide a disposable undergarment that may be worn like traditional underpants but that is absorbent of a woman's menstrual flow.

Another object of this invention is to provide a disposable undergarment, as aforesaid, that is partially disposable in a toilet and partially disposable as solid refuse.

Still another object of this invention is to provide a disposable undergarment, as aforesaid, that includes a moisture resistant layer positioned beneath a removable absorbent layer such that a woman's outer garments are protected from fluids collected on the absorbent layer.

Yet another object of this invention is to provide a disposable undergarment, as aforesaid, that is inexpensive to manufacture and user-friendly to use.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a disposable undergarment according to a preferred embodiment of the present invention;

FIG. 2 is an exploded view of the disposable undergarment as in FIG. 1;

FIG. 3a is an isolated view on an enlarged scale taken from a portion of FIG. 1 illustrating one means for separating the waistband of the outer element;

FIG. 3b is another isolated view as in FIG. 3a illustrating another means for separating the waistband of the outer element;

FIG. 4 is a perspective view from another angle of the inner element of the disposable undergarment as in FIG. 2;

FIG. 5a is a front view of the disposable undergarment as in FIG. 1; and

FIG. 5b is a sectional view taken along line 5b-5b of FIG. 5a.

### DESCRIPTION OF THE PREFERRED EMBODIMENT

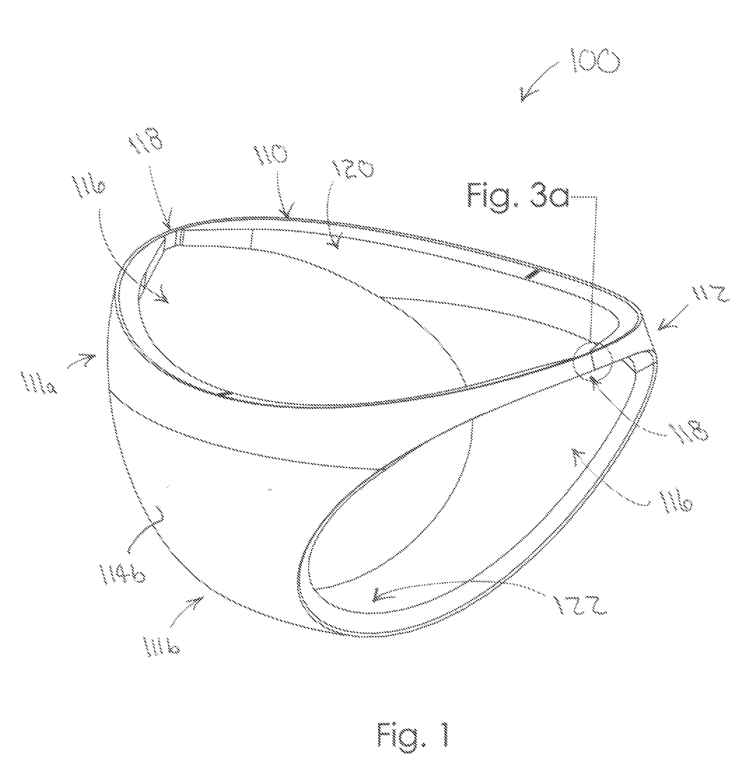

A disposable undergarment 100 according to the present invention will now be described in detail with reference to FIGS. 1 through 5b of the accompanying drawings. More particularly, according to the current invention, disposable underpants 100 include an outer element 110 and an inner element 120.

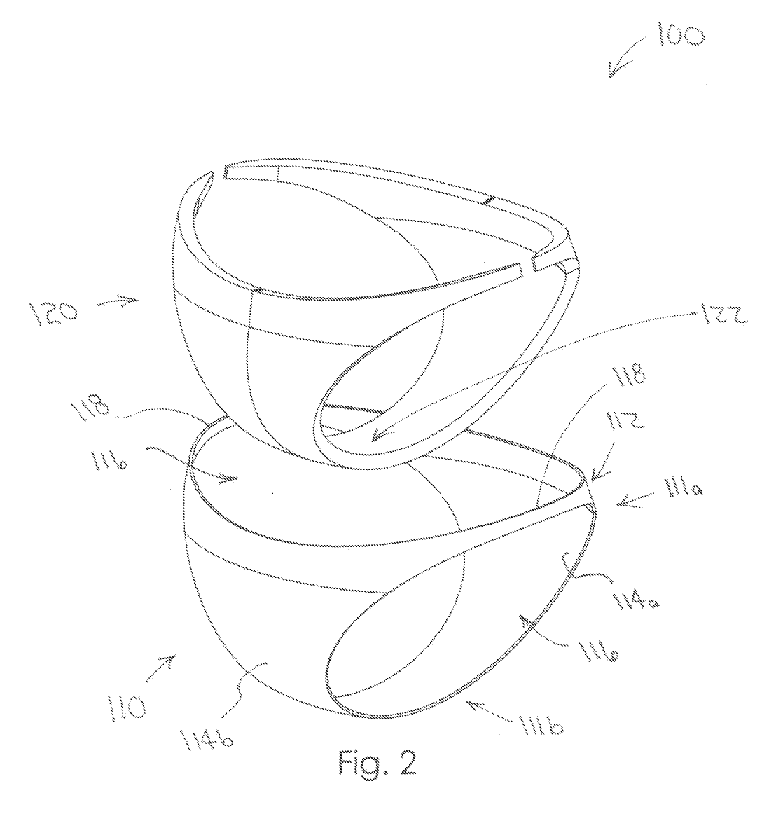

As shown in FIGS. 1 and 2, the outer element 110 has a waistband 112, inner and outer surfaces 114a, 114b, and defines two leg holes 116. More particularly, the outer element 110 may have upper and lower regions 111a, 111b, and the upper region 111a may define the waistband 112; the upper and lower regions 111a, 111b may collectively define the leg holes 116. The waistband 112 has opposed sides 118 upwardly adjacent the leg holes 116. The outer element 110 is disposable as solid refuse, or in other words, includes a material that is unsuitable for disposal in a toilet. For example, the outer element 110 may include a material that does not decompose in the presence of moisture (i.e., a water-resistant material). Either the entire outer element 110 may be constructed of the water-resistant material, or only the lower region 111b of the outer element 110 may be constructed of the water-resistant material.

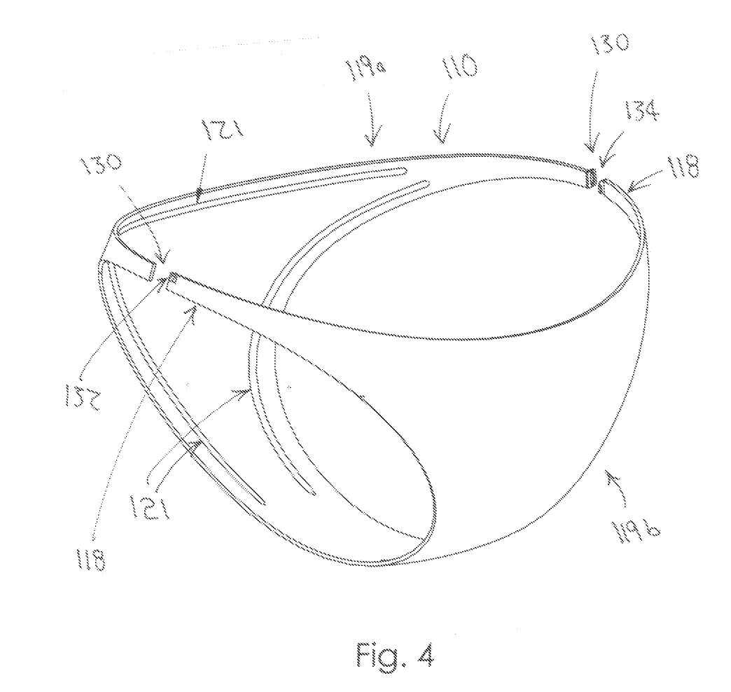

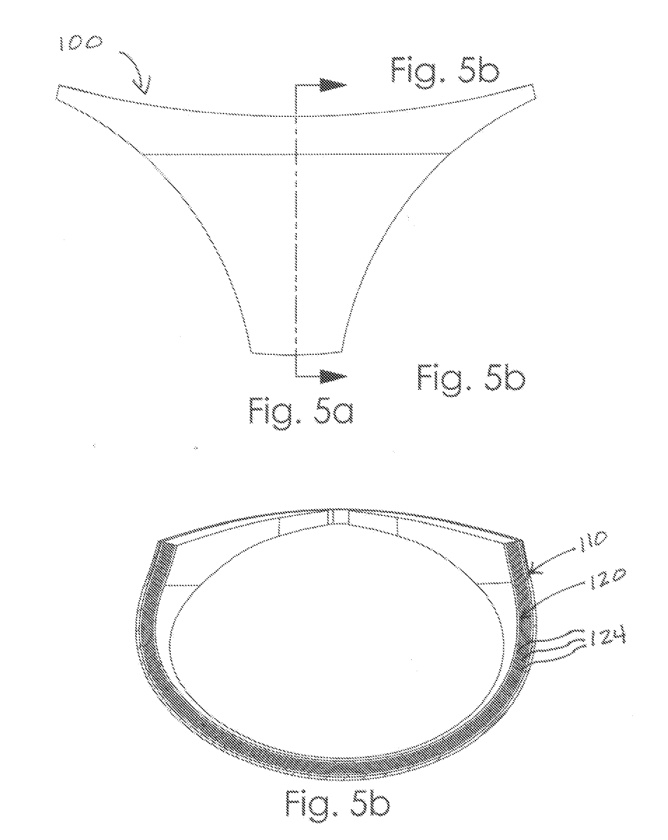

The inner element 120 is removably coupled to the inner surface 114a of the outer element 110, as shown in FIGS. 1 and 2. Adhesive 121 may removably couple the inner element 120 to the outer element 110 (FIG. 4), or other suitable fasteners may be used. The inner element 120 may include a lower region 122 that is adjacent the lower region 111b of the outer element 110 when the inner element 120 is coupled to the outer element 110, and as shown in FIG. 1, the inner element lower region 122 may substantially cover the outer element lower region 111b. While the inner element 110 may cover only the outer element lower region 111b, the inner element 110 may instead substantially cover the entire outer element inner surface 114a as shown in FIG. 1. The inner element lower region 122 may in particular include an absorbent material, and the entire inner element 120 may be constructed of an absorbent material. The inner element 120 is disposable in a toilet, or in other words, is constructed only of material that may decompose in the presence of moisture. As shown in FIG. 5b, the absorbent material may include a plurality of absorbent layers 124 to maximize the absorbent properties while keeping the inner element 120 suitable for disposal in a toilet.

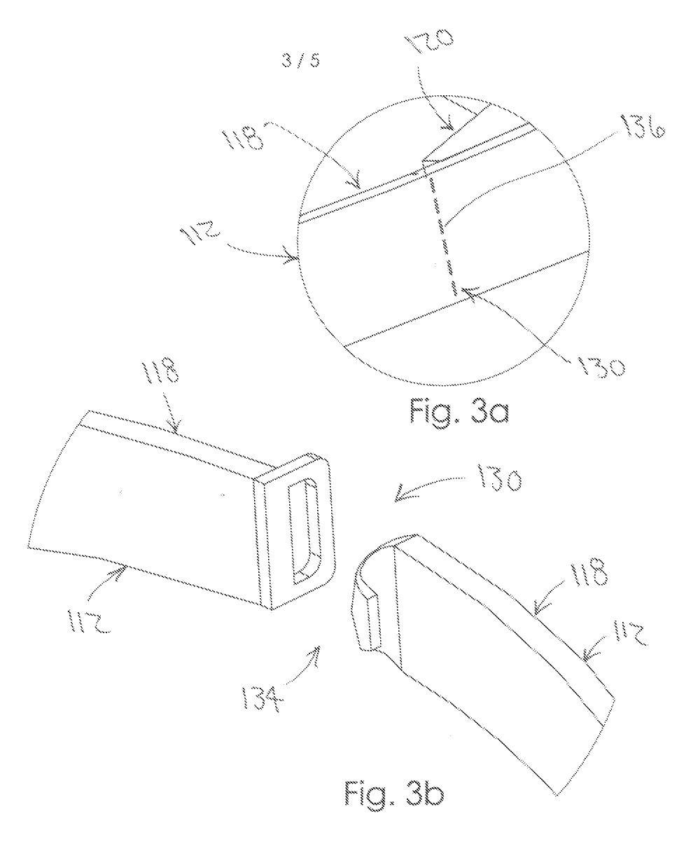

As shown in FIG. 3a through FIG. 4, each waistband side 118 of the outer element 110 may include means 130 for separating the waistband 112 adjacent a respective leg hole 116 to define front and rear portions 119a, 119b of the outer element 110. The means 130 may include, for example, a hook and loop system 132 such as Velcro® (FIG. 4), a clasp system 134 (FIGS. 3b and 4), a perforation 136 (FIG. 3a), and/or other appropriate fasteners. Even if the inner element 120 substantially covers the entire outer element inner surface 114a as noted above, the inner element 120 may not cover the outer element waistband sides 118 upwardly adjacent the leg holes 116; this may allow the inner element 120 to be more easily coupled to and removed from the outer element 110 and/or may allow the outer element 110 to be separated as noted above without hindrance from the inner element 120.

In use, the disposable underpants 100 may be worn under clothing (e.g., pants, dresses, etc.) in a traditional manner. The outer element 110 may provide an attractive appearance similar to prior art underpants. The absorbent nature of the inner element 120 may be used to collect fluids (e.g., due to menstrual flow or incontinence, for example), and the waterproof nature of the outer element 110 may keep those fluids from passing to the clothing. When the inner element 120 becomes wet, the underpants 100 may be removed and the inner element 120 may be separated from the outer element 110 and flushed down a toilet. The outer element 110 may be thrown away in a garbage receptacle. Another pair of disposable underpants 100 may then be worn. The means 130 for separating the waistband 112 adjacent a respective leg hole 116 may allow the disposable underpants 100 to be easily removed (even when wet) and put on. The inner element 120 may be comfortable to the skin, and substantially covering the entire outer element inner surface 114a with the inner element 120 (as discussed above) may allow the outer element 110 to be constructed of materials that are generally uncomfortable to the skin, as the outer element 110 may not contact the skin. Additionally, or alternately, substantially covering the entire outer element inner surface 114a in such a manner may increase the overall attractiveness of the disposable underpants 100 since a consistent thickness may result.

It is understood that while certain forms of this invention have been illustrated and described, it is not limited thereto except insofar as such limitations are included in the following claims and allowable functional equivalents thereof.

1. A disposable undergarment, comprising:
an outer element having a waistband and defining two leg holes, said outer element presenting inner and outer surfaces;
an absorbent inner element removably coupled to said outer element inner surface; and
wherein said inner element is disposable in a toilet and said outer element is disposable as solid refuse.

2. The disposable undergarment as in claim 1, wherein adhesive removably couples said inner element to said outer element.

3. The disposable undergarment as in claim 1, wherein said inner element includes a plurality of absorbent layers.

4. The disposable undergarment as in claim 3, wherein:
said outer element has a lower region; and
said inner element substantially covers said outer element inner surface at said lower region.

5. The disposable undergarment as in claim 1, wherein said outer element includes a water-resistant material.

6. The disposable undergarment as in claim 1, wherein:
said outer element waistband has opposed sides; and
each said side includes means for separating said waistband to define front and rear portions.

7. The disposable undergarment as in claim 6, wherein:
said outer element includes a water-resistant material;
said inner element includes a plurality of absorbent layers; and
adhesive removably couples said inner element to said outer element.

8. The disposable undergarment as in claim 7, wherein said inner element substantially covers said outer element inner surface.

9. The disposable undergarment as in claim 1, wherein:
said outer element waistband has opposed sides; and
each said side includes at least one of a hook and loop system, a clasp system, or a perforation for separating said waistband to define front and rear portions.

10. Disposable underpants, comprising:
an outer element having a waistband, defining two leg holes, and having a lower region, said outer element presenting inner and outer surfaces;
an inner element removably coupled to said outer element inner surface; and
wherein said inner element is disposable in a toilet and said outer element is disposable as solid refuse.

11. The disposable underpants as in claim 10, wherein:
adhesive removably couples said inner element to said outer element;
said outer element lower region includes a water-resistant material;
said inner element has a lower region adjacent said outer element lower region; and
said inner element lower region includes an absorbent material.

12. The disposable underpants as in claim 11, wherein said inner element lower region substantially covers said outer element lower region when said inner element is coupled to said outer element.

13. The disposable underpants as in claim 12, wherein:
said outer element waistband has opposed sides; and
each said side includes means for separating said waistband adjacent a respective leg hole.

14. The disposable underpants as in claim 12, wherein:
said outer element waistband has opposed sides; and
each said side includes at least one of a hook and loop system, a clasp system, or a perforation for separating said waistband adjacent a respective leg hole.

15. Disposable underpants, comprising:
an outer element having upper and lower regions and inner and outer surfaces;
an inner element removably coupled to said outer element inner surface;
wherein said inner element is disposable in a toilet and said outer element is disposable as solid refuse;
wherein said outer element upper region defines a waistband; and
wherein said upper and lower regions of said outer element collectively define two leg holes.

16. The disposable underpants as in claim 15, wherein:
said outer element lower region includes water-resistant material;
said inner element has a lower region adjacent said outer element lower region when said inner element is coupled to said outer element; and
said inner element lower region includes an absorbent material.

17. The disposable underpants as in claim 15, wherein:
adhesive removably couples said inner element to said outer element;
said inner element substantially covers said outer element lower region when said inner element is coupled to said outer element; and
said inner element covers at least a portion of said outer element upper region when said inner element is coupled to said outer element.

18. The disposable underpants as in claim 17, wherein:
said outer element waistband has opposed sides upwardly adjacent said leg holes;
each said side includes means for separating said waistband adjacent a respective leg hole; and
said inner element does not cover said outer element sides upwardly adjacent said leg holes.

19. The disposable underpants as in claim 15, wherein:
said outer element waistband has opposed sides upwardly adjacent said leg holes; and
each said side includes at least one of a hook and loop system, a clasp system, or a perforation for separating said waistband adjacent a respective leg hole.

20. The disposable underpants as in claim 15, wherein:
said outer element waistband has opposed sides upwardly adjacent said leg holes; and
each said side includes means for separating said waistband adjacent a respective leg hole.
